(19) United States
(12) Patent Application Publication
(10) Pub. No.: US 2024/0120039 A1
(43) Pub. Date: Apr. 11, 2024

(54) LookUpTrials

(71) Applicant: Kin Wai Hung, Long Island City, NY (US)

(72) Inventor: Kin Wai Hung, Long Island City, NY (US)

(21) Appl. No.: 18/484,262

(22) Filed: Oct. 10, 2023

Related U.S. Application Data
(60) Provisional application No. 63/378,917, filed on Oct. 10, 2022.

(51) Int. Cl. G16H 10/20

(52) U.S. Cl. CPC G16H 10/20

(57) ABSTRACT

System and method for providing a crowd-sourced knowledge repository and interactive hub for communicating information about clinical trials through an application interface. The application provides access to a database which stores information related to clinical trials and enables authorized users to manage these entries. The application also includes features to facilitate recruitment and further information gathering. Embodiments of the invention are built to provide news and updated information in real-time in a manner that streamlines both the transmission and access aspects for clinical trials. Embodiments of this invention could be applied to similar activities where aggregation of knowledge and efficient recruitment would be desirable, such as event planning across multiple venues or volunteer coordination of numerous projects.

### FIELD OF THE INVENTION

The present invention relates to a system and method for providing and accessing a crowd-sourced knowledge base managed by service providers. More specifically, the present invention is a system and method that utilizes crowd-sourcing techniques and software to support clinical trial search, education, and recruitment. Embodiments of this invention would also be suitable for other activities where a real-time, crowd-sourced knowledge base would be desirable.

### BACKGROUND OF THE INVENTION

Clinical trials are cornerstones to medical innovation, but accrual to clinical trials is becoming more challenging. In oncology, fewer than 1 in 20 adult patients with cancer enroll in clinical trials. A study in 2014 reported that 1 in 4 cancer clinical trials were terminated early with 1 in 10 terminated for poor accrual. Reasons for this poor accrual are multifaceted, though one of the most prominent reasons that medicine is becoming increasingly complex. In this environment, it is becoming more difficult for health care providers to stay aware of up-to-date information about clinical trials. If a health provider is not aware or does not have enough knowledge about the trials, they are less likely to present clinical trial as a treatment option to their patients. Given that over 70% of patients who join a clinical trial report that they did so because they learned about clinical trial options from their providers, there is a critical need to help decrease the cognitive burden for health care providers about relevant clinical trial information at the point of care.

The National Cancer Institute (NCI) provides various resources to support healthcare providers. Most of these resources contain only general information which is not clinically relevant at the point of care. However, there exists a critical need within the clinical trial ecosystem to provide more complex trial information and hidden knowledge to facilitate trial accrual while simplifying and streamlining that information to promote more efficient uptake. Unfortunately, existing solutions, such as public trial registry, have proven inadequate in providing access to quality information at the point-of-care. The present invention bridges this gap by supporting health providers (and patients) with up-to-date, relevant clinical trial information and knowledge to streamline the accrual process across clinical trials networks and beyond. Table 1 offers a comparison between the features of the currently available solutions and the present invention.

### BRIEF DESCRIPTION OF THE DRAWINGS

FIG. 1 is a flow diagram depicting an overview of the system relationships according to an embodiment.

FIG. 2a is a flow diagram depicting an approach to the patient user interface options according to an embodiment.

FIG. 2b is a flow diagram depicting an approach to the study management interface options according to an embodiment.

FIG. 2c is a flow diagram depicting an approach to the provider user interface options according to an embodiment.

FIG. 3 is a flow diagram depicting an approach for the trials listing search according to an embodiment.

FIG. 4 is a flow diagram depicting an approach to modifying the trials database according to an embodiment.

### DETAIL DESCRIPTIONS OF THE INVENTION

All illustrations of the drawings are for the purpose of describing selected versions of the present invention and are not intended to limit the scope of the present invention.

For the purposes of the present invention, several terms are to be understood in a specific context in relation to the invention. The term “patient,” as it relates to users of the system, refers to users whose participation is primarily informational. Patients are enabled to search or access public information, communicate with content managers, and participate in some of the provided social features. The term “participant” refers to patients which have been identified as being a participant in one or more programs within the database. In addition to the accessibility of a patient, participants may be enabled to access general or specific, non-public information within the studies in which they are participating or access elevated communication pathways with study investigators. The term “study investigator” refers to users that are the primary source of content for the system. Study investigators are generally enabled to upload and manage their content, including moderation of the attached social features. The term “provider” refers to institutional users that promote and supplement information about the trials. In addition to the information acquiring elements of the system, providers may be enabled to supplement the available information of a trial, including promotion for individual trials. The term “crowd-source” and its derivatives indicates that the content is provided and primarily managed by user-contributors, rather than the managers and administrators of the invention. “Clinical Trials Pathways” are flow diagrams generated for use in assisting providers and patients in determining whether a trial is relevant to their condition(s), including eligibility standards. Variations of possibilities described for different embodiments should not be construed to be exhaustive unless specifically indicated as such.

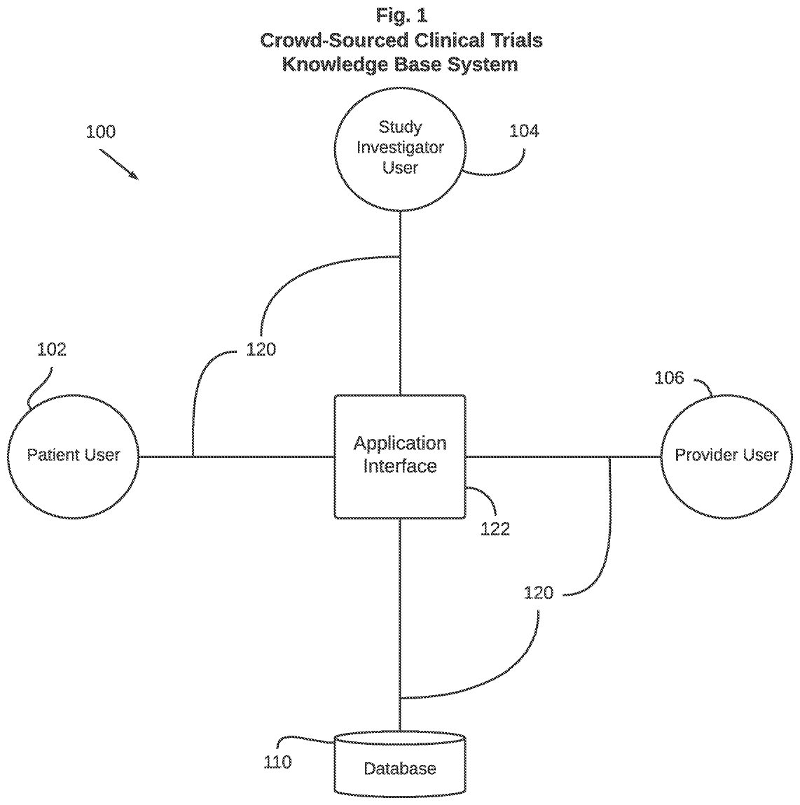

FIG. 1 shows an embodiment for an application accessed knowledge base system 100. The system 100 includes a patient user 102, study investigator user 104, provider user 106, and database 110 interconnected through the linking application 120. Users 102, 104, and 106 are users of the application interface 122. The application interface 122 manages, requests, and displays information from database 110.

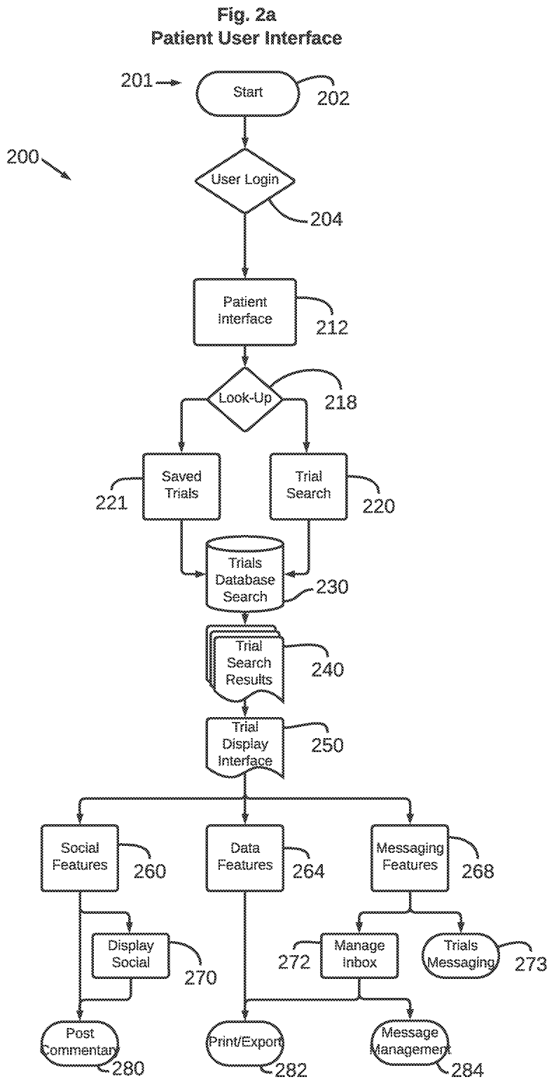

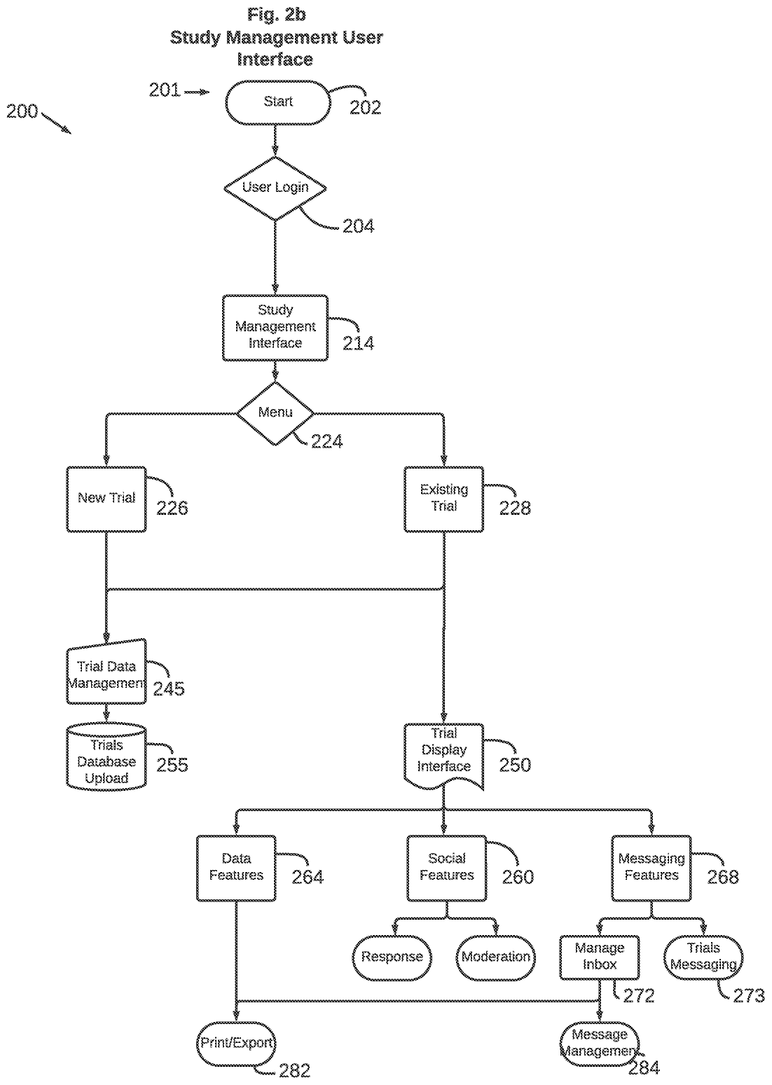

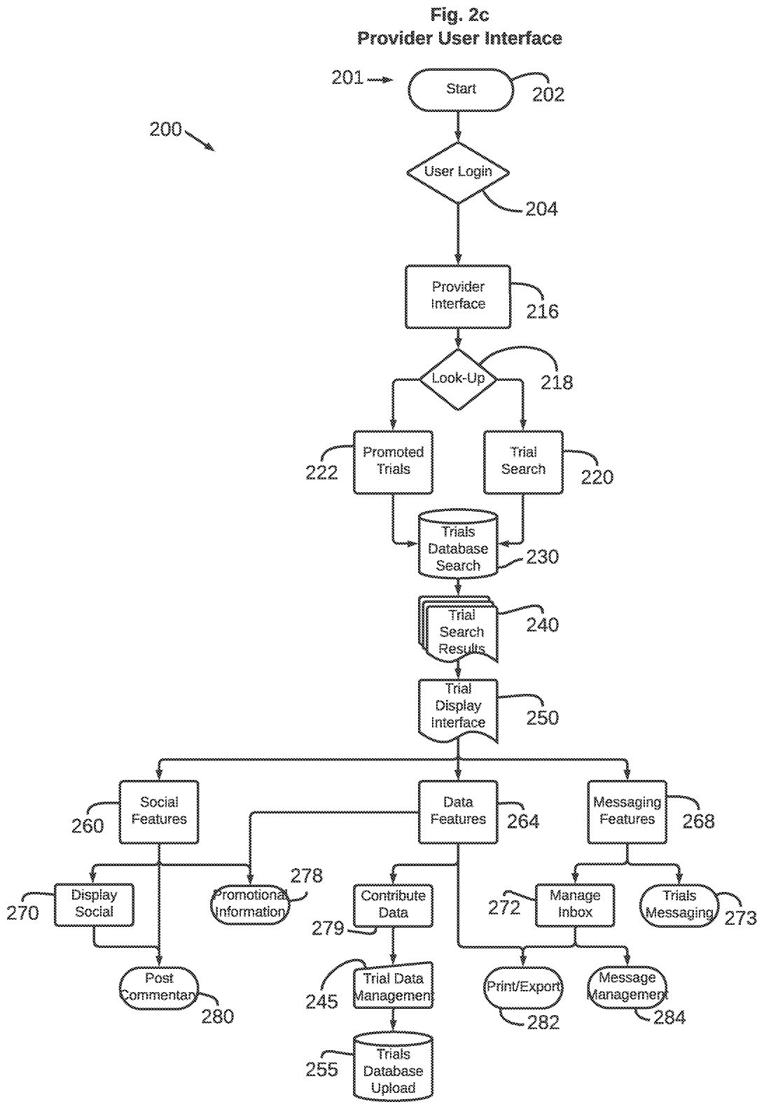

One embodiment of the present invention relates to a system and method for providing and accessing clinical trial information through a software application interface. FIG. 2 is a flow diagram that illustrates one embodiment which provides access to the content based upon a user type. In this embodiment, patients are given the option to initiate a search for trials or retrieve information from a trial they have previously saved or flagged in the application. Other embodiments may enable them to access saved searches or their search history. Embodiments may also allow for voice directed searches, image searching, or through clinical trial pathway diagrams. Different embodiments of the invention may allow a patient to search using keywords, classification tags, location information, symptoms, diagnosis, biomarkers, secondary conditions, or other relevant criteria to identify one or more relevant trials. This process searches the content database for an updated list meeting the search criteria and provides a list for access to the informational interfaces of the individual trials.

Thie Trial Display Interfaces provide public information to users as most recently updated. Embodiments may include text, graphics, audio, and/or video presentation(s) of this information. The information presented may include eligibility criteria, trial title, trial identification number, names of investigators, names of trial coordinators, accrual locations, accrual status, and a trial description. Embodiments may allow for the information to be divided into one or more sections accessible through the interface such as primary trial information, eligibility criteria, trial space availability, news updates, FAQs, or supplemental information. Embodiments of the invention could also allow for further interaction with the trial information such as printing information, exporting data in various file formats, generating personalized clinical trial pathway diagrams, or any provided social and communications features. Embodiments may include social features such as public comments and replies, asking public questions, participating in polls, sharing to through other social applications, or highlighted testimonials. Social features could be presented as dedicated comment sections or fully functional forums in different embodiments. Embodiments may also include communications features such as direct applications to trials, private inquiries, and general messaging. Communications from within the application could remain entirely within the application or provide portals by external formats such as SMS or email.

The embodiment illustrated by FIG. 2 additionally provides options for study investigators to initialize or manage studies. Embodiments of the Trial Data Management interface may provide forms to populate information for new or existing trials. Other embodiments may provide assistance tools for creating, supplementing, or formatting the presentation that will appear in Trial Display Interfaces. Assistance could include options such as templates, machine generated content, file importation, or editing tools. Embodiments may allow for uploading new or updated trials information in real time, on a schedule, with a delay for moderation, or by submission to the system's management. Embodiments may also include access to the social and communications features provided to other types of users. Access to common features may include supplemental access or responsibilities such as social moderation, authority to verify participants, segmenting non-public information, answering questions, or creating polls and other special types of social content. Embodiments could also provide additional data management features such as data analysis tools or exporting user data.

The final user-type illustrated by the embodiment in FIG. 2 is the provider. In this embodiment, providers are enabled to access the same features as those of patients alongside some additional functionality. Providers are also enabled to promote trials through social features or through supplemental data and information. Embodiments may allow for this promotional information to appear as elevated social posts and comments, in location or organization specific sections of the Trials Display Interface, and by being listed as sponsors or promoters within the trials information. Additionally, providers may be enabled to contribute directly to the trials data as a data feature in some embodiments. Data uploaded by a provider may update in real time, require moderation to upload, or be limited to a provider-specific section within the trials database entry. Some embodiments may allow a provider to contribute public and/or non-public information to the trials. Some embodiments may allow for additional communications options including different messaging points of contact than that for patients. Some embodiments may allow a given user to access the system as both a provider and a study investigator. Some embodiments would allow a provider to search on behalf of user or non-user patients and send them the search results or individual trial information through the application's communication features.

Different embodiments of the system could allow for the user interfaces to be accessed through a website as well as a desktop or mobile application. Another embodiment would allow for upload and display of unmanaged trials using their publicly available information. Embodiments may allow for assigning or reassigning the owner or management of a trial.

FIG. 2a, FIG. 2b, and FIG. 2c are a series of flow diagrams demonstrating an overview of the application interface 122 for application 120 for an embodiment of the invention. When the application 120 is accessed 202, the user 201 provides login 204 credentials to indicate whether they are a patient user 102, study investigator user 104, or provider user 106, illustrated by their respective flow diagrams (FIG. 2a, 2b, or 2c). Some embodiments may allow for an anonymous or “guest” patient user 102, whose permissions are limited to reading public information. A patient user 102 is presented with patient interface 212. A study investigator user 104 is presented with study management interface 214. A provider user 106 is presented with provider interface 216. FIG. 2a provides a zoomed in view of FIG. 2, highlighting the patient user flow pattern. FIG. 2b provides a zoomed in view of FIG. 2, highlighting the study management user flow pattern. FIG. 2a provides a zoomed in view of FIG. 2, highlighting the provider user flow pattern. These zoomed in figures otherwise correspond with FIG. 2.

Patient interface 212 and provider interface 216 display a selection where the user 102 or 106 may input search terms (218) to initiate a Trial Search 220 process. Alternately, a patient user may select the option to search from their Saved Trials 221 while a Provider user may select the option to search from their Promoted Trials 222. Each of these selections initiates a Trials Database Search 230 providing an index of Trial Search Results 240. The user 102 or 106 may then select an item from these results, which will be presented in Trial Display Interface 250. Users 102 or 106 may now access Social Features 260, Data Features 264, or Messaging Features 268. Social Features 260 allow for the Social Display 270 or Posting Commentary 280. A provider user 106 may also provide Promotional Information 278. Data Features 264 allow data to be Printed or Exported 282. A provider user 106 may also use Data Features 264 to provide Promotional Information 278 or Contribute Data 279 to the trial itself. Contributing Data 279 initiates the Trial Data Management 245 interface which will allow a Trials Database Upload 255 of the provider contributed data. Messaging Features 268 for user 102 or 106 provide an interface to Manage their Inbox 272 or engage with the Trials Messaging 273 capabilities provided through the application 120. Inbox Management 272 allows a 102 or 106 user to Print or Export 282 information from any messages as well as other Message Management 284 options.

Patient interface 214 displays a selection where the study manager user 104 is shown a Menu 224 where they may elect to provide information for a New Trial 226 or access an Existing Trial 228. Initializing a New Trial 226 or updating an Existing Trial 228 call the process for Trial Data Management 245 to allow for a Trials Database Upload 255. A user 104 accessing an Existing Trial 228 may instead utilize the Trial Display Interface 250 to access the associated Social Features 260, Data Features 264, or Messaging Features 268 associated with the selected Trial. Social Features 260 allow for the Response 275 to or Moderation 280 of promotions and commentary for a trial's linked Social Features 260. Messaging Features 268 for a study manager 104 provide an interface to Manage their Inbox 272 or engage with the Trials Messaging 273 capabilities provided through the application 120. Inbox Management 272 allows a study manager 104 to Print or Export 282 information from any messages as well as other Message Management 284 options.

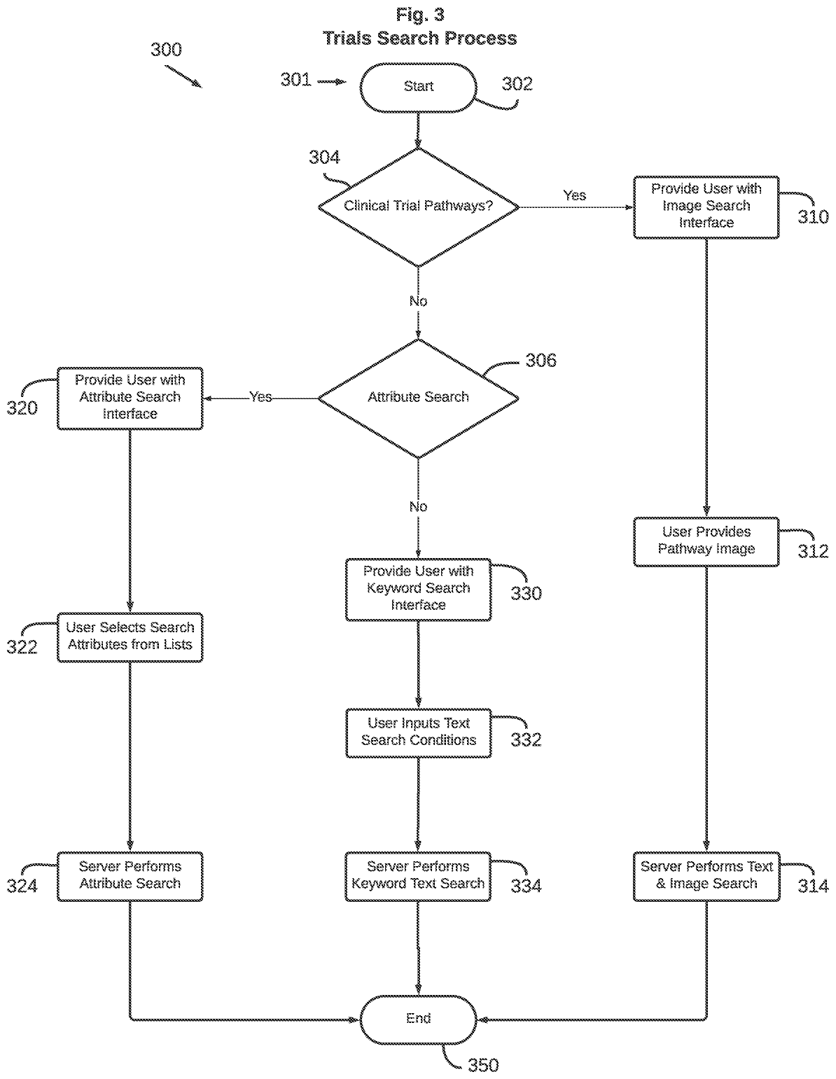

FIG. 3 is a flow diagram demonstrating the search process 300 of an embodiment. When the process is initiated (302) the user 301 indicates whether they wish to search using a Clinical Trials Pathway (304). If the user 301 indicates that they do not wish to search by Pathway (304), they must then indicate whether they wish to perform a Trials Attribute Search (306). If the user 301 declined to perform an Attribute Search (306), then the default Keyword Text Search Interface 330 would be provided to the user 301. From the Keyword Interface 330, user 301 would input Text Query Terms 332 which would initiate a Keyword Search 334 of the whole database. Some embodiments may allow regular expressions (regex) or special search commands to narrow the scope of a Keyword Search 334. If the user 301 indicated that they wished to search by Clinical Trials Pathway (304) then they would be provided with an Image Search Interface 310. User would then upload a Pathway Image 312, allowing a Text and Image Search 314 to be performed. Some embodiments of the Text and Image Search 314 would include a character recognition algorithm, which may utilize adaptive or pre-trained machine learning features, to parse the attributes of the provided Pathway and compare them to those in the database. Some embodiments may also employ an Attribute Search 320 where attributes are recognized from a Pathway. If the user 301 indicated that they wished to perform an Attribute Search (306), they would be provided with the Attribute Search Interface 320. From the Attribute Interface 320, a user 301 would use the provided menus to indicate the Trial Attributes 322 in which they are interested. Selectable attributes may include the specific disease or affliction, progression stage, biomarker status, location, and other relevant trial identifiers. Once selected, an Attribute Search 324 would be conducted. Some embodiments may give value to the attributes, selected by the user 301 or as qualities of the attributes assigned to the trials data, weighting the results to rank trials by relevance to the conditions provided by the user 301. An embodiment may allow a provider user 301 to indicate a number patients under their care needing treatment as an attribute for evaluation their search. Another embodiment may allow a provider or study investigator to search for trials which may be open to additional support or institutional participation that they are in position to provide. Other embodiments may allow the user 301 to select certain attributes as required for display within the search results. The preferred embodiment would combine the Pathway (304) and Attribute (306) decisions, allowing the user 301 to select the Pathway Image Interface 310, Attribute Interface 320, or Keyword Interface 330 from a menu. However, these options are kept separate in the embodiment depicted in FIG. 3 for demonstration purposes. Some embodiments may provide the user with a combined interface, incorporating one or more interfaces (such as 310, 320, 330, or others) then determining the search type from context of supplied conditions which may allow for multiple simultaneous or combined searches. The completion of a search finalizes the process (350), allowing the retrieved results to be provided as a list of selectable entries.

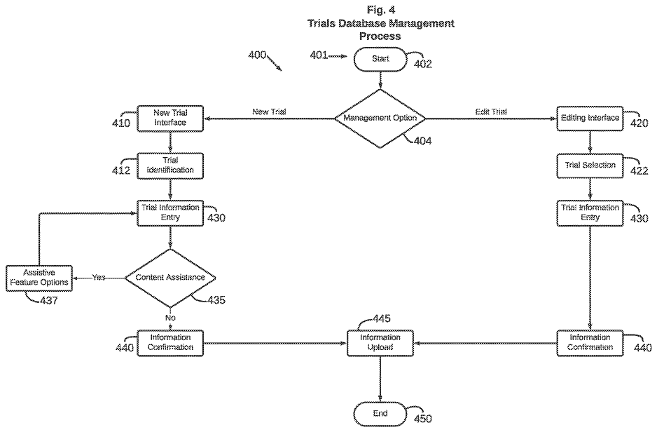

FIG. 4 is a flow diagram demonstrating an embodiment of the Trials Database Management Process 400. When a user 401 initiates the process (402), they are given the option (404) to create a new trial entry or edit an existing one. Creating a new trial displays an interface 410 to facilitate entry into the database. User 401 then provides identifying information for the trial (412). Some embodiments may also generate public or hidden identifying information to facilitate database management. Once the trial entry is initialized, user 401 begins to provide the trial entry's information (430), including presentation and formatting. Some embodiments may allow Trial Identification 412 or Information Entry 430 to be imported from specific files, such as spreadsheets, or from available sources, such as online trial registries. During the Information Entry 430 operations, user 401 may elect to utilize Content Assistance (435) tools to streamline or facilitate the presentation of the entry. Some embodiments may provide templates or context assistive editing features. An embodiment may allow for modification or generation of content using a language or context trained machine learning algorithm. Once a user 401 applies any assistive features (437), they will be given the opportunity to review and modify the results (430) and may then further elect to choose the same or additional assistance (435). Once satisfied, user 401 may confirm the information and presentation (440) to submit the Trial for the Upload Procedure (445). Where editing an existing entry (404) has been elected, user 401 is presented with an Editing Interface 420. User 401 then enters the identifying information to select trial they wish to edit (422). Some embodiments may allow the user to select from a list of trials already associated with the user 401. Some embodiments may allow an authenticated user 401 to begin this editing process 400 from a Display Interface (250). The user 401 is then provided with tools for updating the trials information (430). Some embodiments may provide Content Assistance 435 options when modifying or adding information. One embodiment may allow the user 401 to generate a Clinical Trial Pathway flowchart to be included within the trial's information. Some embodiments may allow the import of additional information through specified file types or other information source options such as trial registry APIs. Once satisfied, user 401 may confirm the information and presentation (440) to submit the Trial for the Upload Procedure (445). In some embodiments, the Upload Procedure (445) may be automatically performed by the server database software upon receipt or at a scheduled time. Other embodiments may allow for a database manager to review the trial entry before scheduling or manually updating the database. Some embodiments may authorize specific users to upload their content automatically while restricting non-authorized users to moderation and review before uploading is performed. Updating the database completes the process (450), enabling the trial to be accessed within the system.

Other embodiments may allow the trial entry to be uploaded without publication, limiting access to specified users for review or generally restricting the information to specified users such as providers and participants. Some embodiments may allow for user 401 to be an automated agent that serves as a manager for unmanaged trials or a supplemental manager for managed trials. In some embodiments, an automated user 401 could be enabled to schedule updates from external source information. Other embodiments may allow for editing privileges to be limited for a specific user 401, by user type or other criteria. One embodiment of a restricted user 401 could allow for a provider to supplement the trial information in an area that would be displayed to identify that information as specific to that provider's organization or location. Other embodiments may allow for a restricted participant user 401 to be enabled to provide blog entries or context outside of the social features.

A search will usually return a list of trials for a user to browse. An embodiment may skip the list display when a search only returns a single result, instead displaying that trial as if selected from the list. Some embodiments may display the list of search results showing the trials' titles. Other embodiments may include short summaries of information, such as those found when using short-form social media applications. Some embodiments may highlight entries based on relevance, news, or other criteria. Other embodiments may show more or less summary information of an entry within the list based on those or other criteria. Some embodiments may allow the list to be sorted according to single or multiple selectable criteria such as duration, age, eligibility, recruitment status, or affliction. Some embodiments may allow a user to choose a preferred amount of data presented from the list display. Some embodiments may allow some visual or audio media to be embedded within a list entry, making some media accessible without fully selecting a given trial.

When presenting the information of selected trials, one embodiment may display a trial's information on a single page with segmented information delineated visually, such as placing lines between sections. Other embodiments may allow for a trial's information to be split into multiple linked pages. Some embodiments may restrict certain information as non-public, allowing only specific users access. Other embodiments may highlight specific information such as recruiting status or news. Some embodiments may allow audio or video media to be embedded within or linked to static information sections. One embodiment would allow the trial information to be printed or exported to a selection of file formats such as a spreadsheet or graphical format. Some embodiments may provide the ability to generate a Clinical Trials Pathway flowchart for print or export. Other embodiments may have specific information available to print or export which may include or exclude designated information to enhance the file or print formatting. Another embodiment may allow for user specific non-public information to be eligible as print or export options. Some embodiments may allow patients to apply to trials directly from the display interface, either within the application interface or by linking to external form(s).

An embodiment may also include a variety of social features within a Trial's Display Interface. An embodiment may store the social information as part of a trial's database entry. Other embodiments may store or process the social features using a separate database or social media system. Some embodiments may provide a user forum such as a bulletin board system or something similar to a chosen social media model. In some embodiments, social features could be divided into sections such as one for asking questions or another for experiential commentary. Embodiments may enable certain user types or specific users with moderation and administrative capabilities. One embodiment may place moderation authority with host or promoting providers. Some embodiments may associate all or a selection of the social features with a provider profile such as having a forum space dedicated for client-patients of a provider organization. Some embodiments may allow a provider to promote a trial for a specified purpose such as informing users that the organization is providing all or part of the trial facilities, accrual and recruitment for the trial, or promising preliminary results.

An embodiment may provide communications and messaging features to users within the application interface. Embodiments may allow for communication with other users. Some embodiments may limit the communication between specific users such as by restricting queries to study investigators to options through specific provided forms. One embodiment may format those communications to utilize formats enabling cross communication with stakeholders outside of the system. Some embodiments may duplicate communications to facilitate in-application messages and external messaging simultaneously or as a contingency. Embodiments may allow for message management options similar to a email or private messaging interface. Some embodiments may assign priority or urgency indicators to messages based on the users type or status.

While the described embodiments refer to clinical trials, embodiments of the present invention could be adapted to facilitate similarly crowd-sourced and operated knowledge repositories of unrelated activities. One embodiment could apply the system and method to event planning, where venues and event organizers can interact with attendees. Another embodiment could utilize the invention for marketing or other types of academic research, particularly where such research requires detailed knowledge to make participation and recruitment more efficient. The described embodiments should not be construed to limit the invention to specific activities, such as clinical trials.

TABLE 1
Comparison of LookUpTrials with Various “Trial-Recruitment” Technologies

| | Features (✓: yes; +/−: sometimes) | | | | |
|---|---|---|---|---|---|
| | | | Trial | | |
| | | | Matching | Trial | |
| | Trial Registry | | (e.g., | recruitment | |
| | (e.g., | | Watson, | app (e.g., | |
| | ClinicalTrials.Gov) | EHR | MatchMiner) | TrialX) | LookUpTrials |
| Trial Search (Include AI- | ✓ | ✓ | ✓ | ✓ | ✓ |
| enabled) | | | | | |
| Curated Clinical Trial | ✓ | ✓ | ✓ | ✓ | ✓ |
| Content* | | | | | |
| *Managed by Clinical | | +/− | +/− | +/− | ✓ |
| Trial Team | | | | | |
| Real-Time Update and | | | | +/− | ✓ |
| Editing | | | | | |
| Knowledge Sharing | | | | +/− | ✓ |
| Forum | | | | | |

1. A system for facilitating a crowd-sourced knowledge repository and delivery of repository information, the system comprising:
a server coupled to one or more components containing one or more databases, one or more search engine components, one or more indexing components, and an authentication component;
an application capable device coupled to the server via a network and having a microprocessor component, a storage device, a display device for presenting a graphical user interface, and one or more input components which allow a user to create or interact with knowledge-base information, and the authentication component on the server and one or more application modules;
wherein the knowledge-base information comprises of one or more database entries to be searched and indexed, one or more attributes for each database entry, one or more user accounts, and a search engine;
the search engine delivered as a software service over the network, generated according to search conditions received from the application capable device and configured to search the one or more database entries according to the received search conditions;
the search engine is configured to receive at least one search condition comprising of at least one attribute or keyword and retrieve database entries according to the received search conditions;
the search engine is configured as a module of the search engine component on the server;
the one or more databases configured to store the one or more database entries, one or more attributes for each database entry, and one or more user accounts;
the authentication component configured to receive identifying information from the application, identify a user according to identifying information received, and provide permission for access to the features enabled by their type of user account;
the one or more indexing components configured to store the database entries retrieved by the search engine and index the entries into a format viewable by the application capable device;
a display module configured to display search results, selected database entries, and selectable features on the display device;
a selection module configured to receive input for selecting a database entry from among the displayed search results;
the selection module configured to receive input for selecting accessible features provided by modules that are displayed within the interface;
a data management module configured to give the user options to export or print information from a selected database entry;
a social features module configured to display and enable interaction with social features linked to a selected database entry;
a messaging module configured to facilitate communication with the users associated with a selected database entry and manage received messages;
wherein the selection module, data management module, social features module, and messaging module are application modules configured to be processed through the microprocessor;
a database update module configured to receive database entry information and attributes from the user to create or update a database entry;
wherein the database update module is an application module configured to be processed through the microprocessor and transmit the information to the one or more databases; and
the one or more databases configured to receive database entry information from the database update module over the network.

2. The system of claim 1, wherein the application capable device is a phone or tablet device.

3. The system of claim 1, wherein:
a file upload module is configured to accept files as user input;
the file upload module is an application module configured to be processed through the microprocessor and store uploaded files on the storage device;

4. The system of claim 3, wherein the file upload module is further configured to process images input as search conditions, derive attributes from the processed image, and provide the derived attributes to the search engine in place of the image as the user's search conditions.

5. The system of claim 4, wherein the image file is a Clinical Pathway Flowchart.

6. The system of claim 3, wherein the database update module is configured to import a file uploaded through the file upload module as information for creating or updating database entries.

7. The system of claim 1, wherein the selection module is further configured to automatically select a retrieved database entry when the search engine only retrieves a single entry.

8. The system of claim 7, wherein:
the data management module is further configured to provide a user with the option to select one or more database entries as favorites and store unique identifying information of the selected one or more database entries in the storage device;
the search engine is further configured to directly retrieve a database entry when a user provides unique identifying information as a search condition;

9. The system of claim 1, wherein the database update module is configured to receive API data through the network as information for creating or updating database entries.

10. The system of claim 1, wherein the messaging module is configured to send messages externally over the network.

11. The system of claim 1, wherein the database entries are stored clinical trial information.

12. A method for facilitating a crowd-sourced knowledge repository and delivery of repository information, the method comprising:
providing a server coupled to one or more components containing one or more databases, one or more search engine components, one or more indexing components, and an authentication component;
providing an application capable device coupled to the server via a network and having a microprocessor component, a storage device, a display device for presenting a graphical user interface, and one or more input components which allow a user to create or interact with knowledge-base information, and the authentication component on the server and one or more application modules;
wherein the knowledge-base information comprises of one or more database entries to be searched and indexed, one or more attributes for each database entry, one or more user accounts, and a search engine;
providing the search engine delivered as a software service over the network, generated according to search conditions received from the application capable device and configured to search the one or more database entries according to the received search conditions;
receiving at least one search condition comprising of at least one attribute or keyword by the search engine and retrieving database entries according to the received search conditions;
building the search engine as a module of the search engine component on the server;
storing the one or more database entries, one or more attributes for each database entry, and one or more user accounts within the one or more databases;
receiving identifying information from the application, identifying a user according to the identifying information received, and providing permission for access to the features enabled by their user account by the authentication component;
indexing, in the one or more indexing components, to store the database entries retrieved by the search engine and index the entries into a format viewable by the application capable device;
building a display module to display search results, selected database entries, and selectable features on the display device;
building a selection module to receive input for selecting a database entry from among the displayed search results;
receiving input for selecting accessible features provided by modules that are displayed within the interface in the selection module;
building a data management module to give the user options to export or print information from a selected database entry;
building a social features module to display and enable interaction with social features linked to a selected database entry;
building a messaging module to facilitate communication with the users associated with a selected database entry and manage received messages;
wherein the selection module, data management module, social features module, and messaging module are application modules processed by the microprocessor;
building a database update module to receive database entry information and attributes from the user to create or update a database entry;
wherein the database update module is an application module using the microprocessor to transmit the new or updated information to the one or more databases; and
building the one or more databases to receive database entry information from the database update module over the network.

13. The method of claim 12, wherein the provided application capable device is a phone or tablet.

14. The method of claim 12, building a file upload module as an application module of the microprocessor to upload files and store them on the storage device.

15. The method of claim 14, building the file upload module to process images input as search conditions, derive attributes from the processed image, and provide the derived attributes to the search engine in place of the image as the user's search conditions.

16. The method of claim 14, building the database update module to import files uploaded through the file upload module as information for creating or updating database entries.

17. The method of claim 12, building the database update module to receive API data through the network as information for creating or updating database entries.

18. The method of claim 12, building the messaging module to send messages externally over the network.

19. A system for facilitating a crowd-sourced repository of clinical trials and delivery of repository information through a remote application, the system comprising:
a server coupled to one or more components containing one or more databases, one or more search engine components, one or more indexing components, and an authentication component;
an application capable device coupled to the server via a network and having a microprocessor component, a storage device, a display device for presenting a graphical user interface, and one or more input components which allow a user to create or interact with knowledge-base information, and the authentication component on the server and one or more application modules;
wherein the graphical user interface is a remote application configured to accept input from a user through the input components, display output of the system on the display device, processing the one or more application modules through the microprocessor component, and communicating with the server over the network;
wherein the knowledge-base information comprises of one or more database entries to be searched and indexed, one or more attributes for each database entry, one or more user accounts, and a search engine;
the search engine delivered as a software service over the network through the application, generated according to search conditions received from the remote application and configured to search the one or more database entries according to the received search conditions;
the search engine is configured to receive at least one search condition comprising of at least one attribute or keyword and retrieve database entries according to the received search conditions;
the search engine is configured as a module of the search engine component on the server;
the one or more databases configured to store the one or more database entries, one or more attributes for each database entry, and one or more user accounts;
the authentication component configured to receive identifying information from the remote application, identify a user according to identifying information received, and provide permission for access to the features enabled by their user account;
the one or more indexing components configured to store the database entries retrieved by the search engine and index the entries into a format viewable by the remote application;
a display module configured to display search results, selected database entries, and selectable features on the display device;
a selection module configured to receive input for selecting a database entry from among the displayed search results;
the selection module configured to receive input for selecting accessible features provided by modules that are displayed within the interface;
a data management module configured to give the user options to export or print information from a selected database entry;
a social features module configured to display and enable interaction with social features linked to a selected database entry;
a messaging module configured to facilitate communication with the users associated with a selected database entry and manage received messages;
wherein the selection module, data management module, social features module, and messaging module are application modules configured to be processed through the remote application;
a database update module configured to receive database entry information and attributes from the user to create or update a database entry;
wherein the database update module is an application module configured to be processed through the remote application and transmit the information to the one or more databases; and
the one or more databases configured to receive database entry information from the database update module over the network.

20. The system of claim 19, wherein the remote application is a web application accessed and controlled through an internet browser interface.
